(19) United States
(12) Patent Application Publication
(10) Pub. No.: US 2012/0286038 A1
(43) Pub. Date: Nov. 15, 2012

(54) SANITARY DISPENSING DEVICE WITH REMOVABLE DISPLAY

(76) Inventor: Ming-Yuan WU, Taipei City (TW)

(21) Appl. No.: 13/106,522

(22) Filed: May 12, 2011

(51) Int. Cl. G07F 9/00; A61L 2/10; G09F 3/00; G07F 7/04; G07F 11/00

(52) U.S. Cl. 235/381; 221/155; 194/206; 40/299.01; 250/492.1

(57) ABSTRACT

A sanitary dispensing device for dispensing and storing products is disclosed. The sanitary dispensing device comprises a product display area for displaying products or pictures of products, product selection switches for selecting a product for purchase, a payment exchange area for receiving payment, and a product retrieval area for retrieving purchased products. The sanitary dispensing device further comprises a removable display for displaying information such as product information, instructional information, or dynamic advertising. The sanitary dispensing device further comprises an ultraviolet light module for performing sterilization procedures on the products, product packages, and currency. The sanitary dispensing device further comprises a hands sterilization module for sterilizing a user's hands.

### BACKGROUND OF THE INVENTION

1. Field of the Invention

The present invention relates to dispensing equipment. More specifically, the present invention discloses a device for storing and dispensing products and sterilizing the products prior to dispensing in order to ensure suitable sanitary conditions of the purchased goods and currency exchanged.

2. Description of the Prior Art

A convention vending machine is a convenient way for a user to make a purchase of a small item. A user inserts coins into the vending machine and receives their purchased item.

However, the conventional vending machine provides limited interaction with the user. The machine simply accepts the coins and dispenses the product. As a result, users are indifferent to the machine and fail to receive a personalized experience from the exchange.

Additionally, the conventional vending machine stores products that can easily get dirty or unsanitary from bacteria while stored in the machine. The user of the conventional vending machine receives a product with potentially harmful conditions.

Furthermore, all money entering the vending machine and money given to the user as change is also contaminated with bacteria and germs and as the money circulates more and more people are affected.

As a result, the conventional vending machine provides conditions that are unsanitary to users and can negatively affect a user's health.

Therefore, there is need for a safe and efficient device which dispenses products and ensures goods, packaging, a currency are are sterile and sanitary.

### SUMMARY OF THE INVENTION

To achieve these and other advantages and in order to overcome the disadvantages of the conventional methods in accordance with the purpose of the invention as embodied and broadly described herein, the present invention provides a device for storing and dispensing products and ensures suitable sanitary conditions of the purchased goods or packaging.

The sanitary dispensing device of the present invention dispenses products that are relatively inexpensive such as, for example, food items, snacks, beverages, cosmetic supplies, household items, etc.

The sanitary dispensing device also allows users to purchase expensive items not stocked in the dispensing device. The user is issued a coupon that the user redeems for the product at a store or distribution area.

The sanitary dispensing device of the present invention comprises a product display area, product selections switches or buttons, a payment exchange area, and a product retrieval area.

The product display area displays products, samples of products, or pictures of products that are available for purchase.

The product selection switches are for selecting a product or goods for purchase. The user presses the associated product selection switch for the item they want to purchase.

The payment exchange area is for receiving payment via coin currency, paper currency, or debit card. The payment exchange area also displays the cost of the selected product, amount owed for purchase, or amount already received. A keypad is also provided for entering product number or code.

The products are retrieved by the user from the product retrieval area after purchase.

The dispensing device further comprises a removable display for displaying information such as product information, instructional information, promotional video, or dynamic advertising. The removable display can be added to or removed from the dispensing device according to requirements.

The sanitary dispensing device further comprises an ultraviolet light module for performing sterilization procedures on the products or product packaging. This further ensures that the items are sterilized and increases consumer confidence in the products. Since the ultraviolet sterilization procedure is fast, effective, and inexpensive the ultraviolet light module of the present invention provides an efficient method of ensuring sanitary conditions of the goods or packaging.

The sanitary dispensing device of the present invention further comprises an ultraviolet light module for performing sterilization procedures on paper currency and coin currency that is input into the sanitary dispensing device. An ultraviolet light module is also provided for sterilizing currency that is to be provided to the user as change from a purchase.

A hands sterilization module is provided for sterilizing a user's hands. The user places their hands into a hand sterilizing area and a sterilizing gel or liquid is sprayed onto the user's hands thereby sterilizing the user's hands.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings:

FIG. 1 is a drawing illustrating a sanitary dispensing device according to an embodiment of the present invention;

FIG. 2 is a drawing illustrating a payment exchange area of a sanitary dispensing device according to an embodiment of the present invention;

FIG. 3 is a drawing illustrating a sanitary dispensing device with removable display according to an embodiment of the present invention;

FIG. 4 is a drawing illustrating a sanitary dispensing device with an ultraviolet light sterilization module according to an embodiment of the present invention;

FIG. 5 is a drawing illustrating a sanitary dispensing device with an ultraviolet light sterilization module and sterilizing area according to an embodiment of the present invention;

FIG. 6 is a drawing illustrating a payment exchange area of a sanitary dispensing device according to an embodiment of the present invention;

FIG. 7A is a drawing illustrating a sanitary dispensing device with a hands sterilization module with sterilizing area according to an embodiment of the present invention;

FIG. 7B is a drawing illustrating a hands sterilization module with sterilizing area according to an embodiment of the present invention;

FIG. 8A is a drawing illustrating a sanitary dispensing device with a coin currency input ultraviolet light sterilization module according to an embodiment of the present invention;

FIG. 8B is a drawing illustrating a sanitary dispensing device with a paper currency input ultraviolet light sterilization module according to an embodiment of the present invention; and

FIG. 8C is a drawing illustrating a sanitary dispensing device with a change return ultraviolet light sterilization module according to an embodiment of the present invention.

### DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

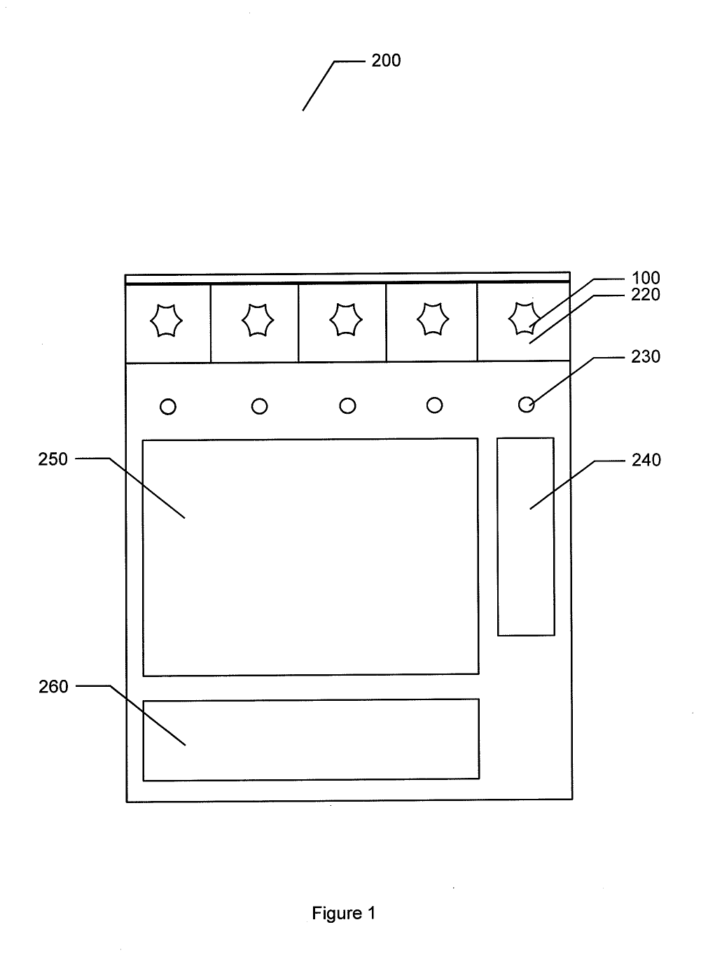

Refer to FIG. 1, which is a drawing illustrating a sanitary dispensing device according to an embodiment of the present invention.

As shown in FIG. 1, the sanitary dispensing device 200 comprises a product display area 220, a plurality of product selection switches 230, a payment exchange area 240, and a product retrieval area 260.

The product display area 220 comprises a picture or photograph of the product 100, a sample of the product 100, or an actual product 100 available for purchase. For example, a picture of the product 100 is provided so the user can identify which item they wish to purchase or obtain. Or, for example, a sample of the product 100 is provided and when the user selects a sample, the actual product purchased is obtained from the product retrieval area 260 and the product sample remains in the product display area 220. Or, for example, the actual product 100 is displayed in the product display area 220 and when selected, the actual product 100 drops into the product retrieval area 260 and a new actual product 100 is displayed in the product display area 220.

In an embodiment of the present invention the product display area 220 further comprises an indication of the cost of the product.

In an embodiment of the present invention the sanitary dispensing device 200 further comprises a product storage area 250 for storing an inventory of products 100. After a user submits payment for the desired product 100, one of the appropriate products stored in the product storage area 250 drops into the product retrieval area 260 and the user can then retrieve the purchased item 100.

In an embodiment of the present invention the product display area 220 fulfills the purpose of the product storage area 250 since the retrieved product 100 is an actual product 100 for purchase. An inventory of actual products are stored and displayed in the product display area 220.

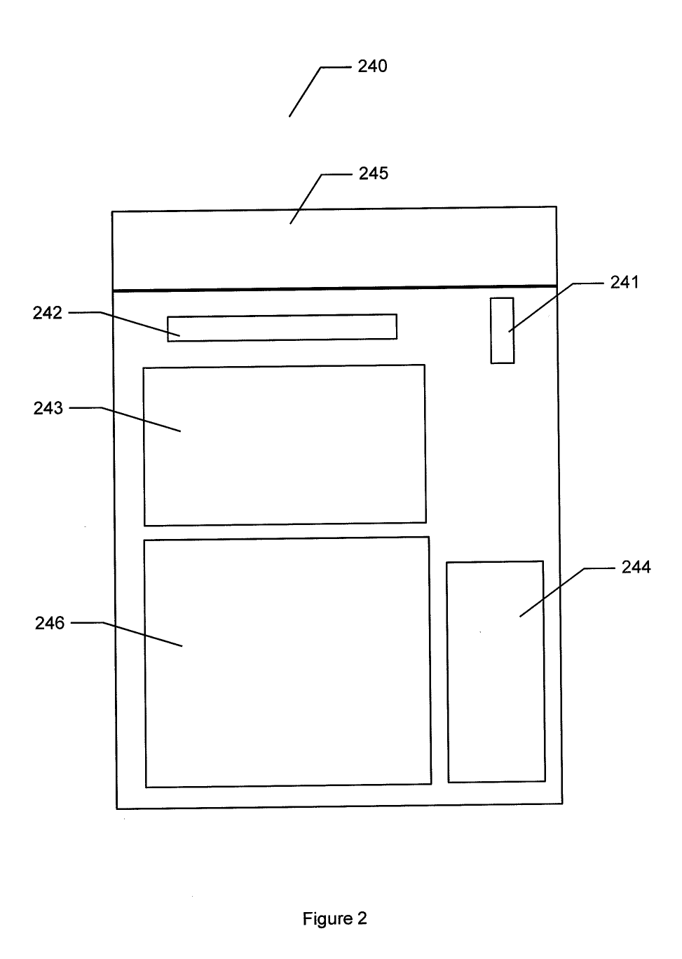

Refer to FIG. 2, which is a drawing illustrating a payment exchange area of a sanitary dispensing device according to an embodiment of the present invention.

The payment exchange area 240 enables exchanges of payment for purchases of products between users and the dispensing device.

In the embodiment illustrated in FIG. 2, the payment exchange area 240 comprises a payment display 245, a coin currency input 241, a paper currency input 242, and a change return 244.

The payment exchange area 240 further comprises a payment exchange controller for interfacing between and controlling the payment display 245, the coin currency input 241, and the paper currency input 242.

The coin currency input 241 allows users to insert coins into the coin currency input 241 of the dispensing device in order to make payment for a selected product. This is convenient for users when the cost of a particular product is relatively inexpensive. The coin currency input 241 comprises means for accepting coins that are input by the user, detecting the denomination of the coins, storing the coins, providing coins to the change return 244 when change is due the user, and informing the payment exchange controller of the denomination of a received coin.

Similarly, the paper currency input 242 allows users to make payment with paper currency. This payment method is more efficient when purchasing products that are more expensive and reduces the effort required instead of purchasing using coins. The paper currency input 242 comprises means for accepting paper currency that is input by the user, detecting the denomination of the paper currency, storing the paper currency, and cooperates with the payment exchange controller regarding the denomination of received paper currency.

The change return 244 receives appropriate coins for returning change to the user after purchasing a product and allows the user to retrieve their change.

The payment display 245 is, for example, a small LCD or LED display. The payment display 245 shows the amount of money owed in order to purchase a selected product, the amount of money received, or both the amount of money owed and amount of money received. As each coin or piece of paper currency is received, the payment display 245 is updated to reflect the current amount of money received and/or owed.

In an embodiment of the present invention the payment exchange area 240 further comprises a keypad 246 for entering product selection data. In this embodiment an indicator of the product number for each product is displayed in the product display area 220 and the keypad 246 acts as the product selection switches 230. The user identifies which product they want to purchase by noting the product number in the product display area 220 and enters the product number using the keypad 246. The payment exchange controller detects the entered product number and displays the amount of money required to purchase the product on the payment display 245.

In an embodiment of the present invention the payment exchange area further comprises a card reader/writer 243 for receiving payment from a debit card or reading user information from a membership card. The card reader/writer 243 and/or the payment exchange controller determines how much money is owed for the purchase, determines if the debit card has sufficient funds to cover the purchase, and deducts the amount owed from the funds stored in the debit card. The card reader/writer 243 also reads user information from a membership card. After reading the user's information the card reader/writer 243 and/or the payment exchange controller determines various aspects of the status or the user. For example, what level of membership the user has, what pricing or discounts to offer, determines how many points the user has, determines a user's past purchase history, adds points to a user's membership card or account upon purchases, etc.

For example, if the user has a membership card with an available number of premium points, the user can exchange the points for a product.

Also, for example, a user with a membership card with a certain level ranking/rating, the user is offered a discount price or a price at a lower cost than a user without a membership card or a user with a membership card with a lower level of ranking/rating.

If a personal identification number (PIN) or password is required in order to use the debit card or membership card, the PIN or password is entered via the keypad 246.

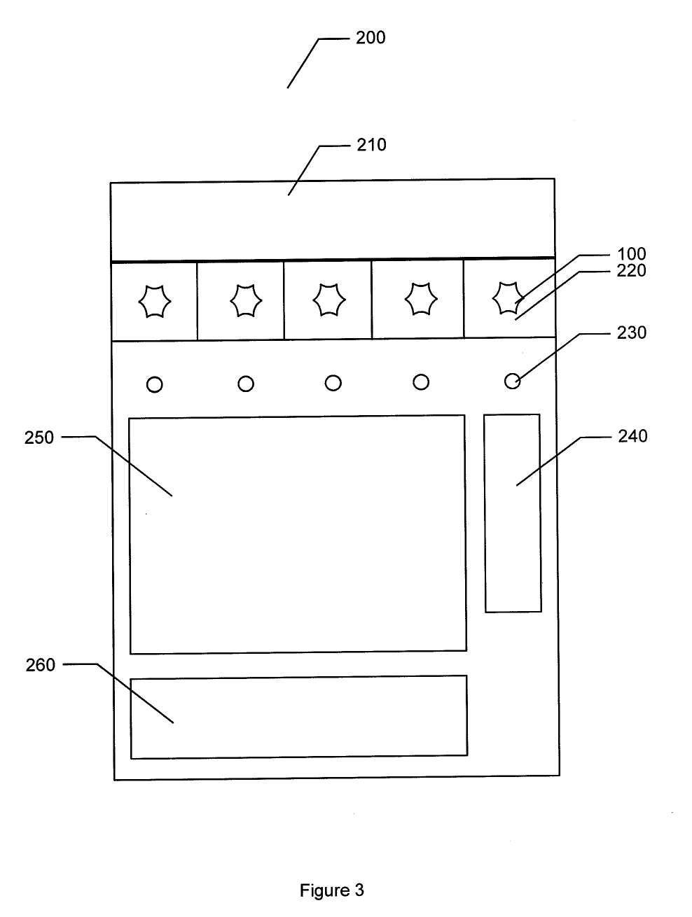

Refer to FIG. 3, which is a drawing illustrating a sanitary dispensing device with removable display according to an embodiment of the present invention.

The embodiment of the present invention illustrated in FIG. 3 is similar to the embodiment of FIG. 1. However, in the embodiment illustrated in FIG. 3 the sanitary dispensing device 200 further comprises a removable display 210.

The removable display 210 can be added to or removed from the dispensing device 200 to increase flexibility of application or use.

The removable display 210 allows the dispensing device 200 to display information such as, for example, product information, instructional information, advertising, or a combination of different types of information. In an embodiment of the present invention the removable display 210 is an electronic display that is capable of displaying video or multimedia presentations.

An application of the removable display 210 is displaying advertisements of the products available for purchase from the dispensing device 200. Another application of the removable display 210 is displaying instructional videos of how to use the products. Another application of the removable display 210 is displaying promotional videos of how the products are manufactured or the benefits and advantages of the products.

Although the removable display 210 can be removed, the removable display is secured in a manner in order to prevent theft of the removable display 210.

The removable display 210 can be added or removed according to application requirements or cost concerns.

The removable display 210 also allows dynamic advertising to be provided to users. For example, when a user selects a product to purchase the removable display 210 displays advertising for competing products or displays products that would be suitable for the user in conjunction with their selected item. For example, if the user selected a food item to purchase, an advertisement for a suitable beverage is provided to entice the user to make an additional purchase.

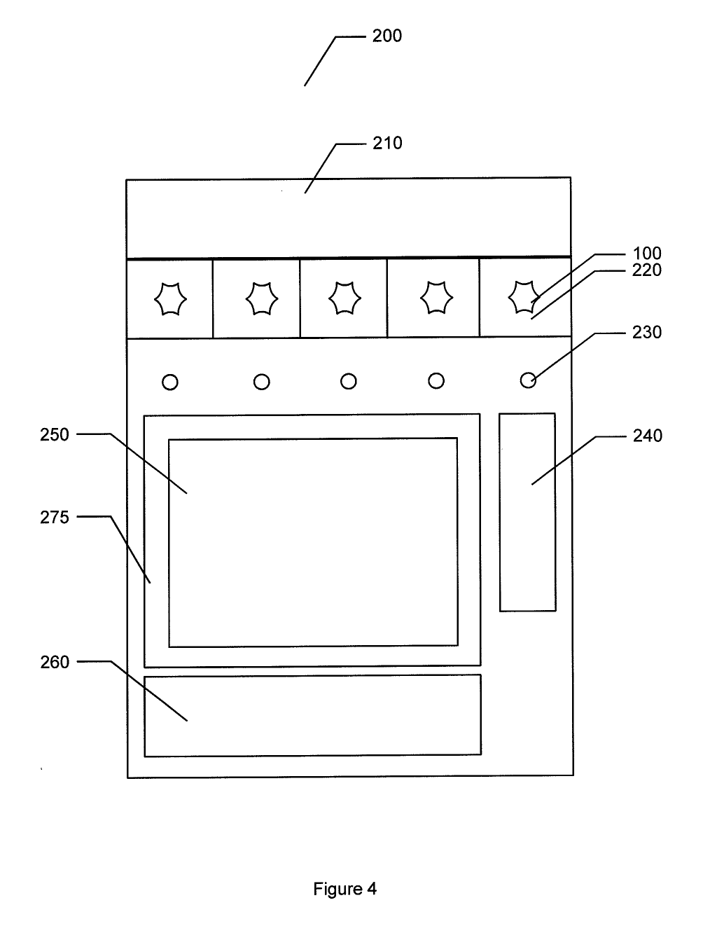

Refer to FIG. 4, which is a drawing illustrating a sanitary dispensing device with an ultraviolet light sterilization module according to an embodiment of the present invention.

In the embodiment illustrated in FIG. 4, the sanitary dispensing device 200 further comprises an ultraviolet light module 275. In this embodiment the ultraviolet light module 275 is provided for performing sterilization procedures on the products or product packaging stored in the product storage area 250.

The ultraviolet light module 275 comprises a lamp or a plurality of lamps, for example, a mercury-vapor lamp that emits ultraviolet light at the germicidal wavelength.

The ultraviolet light module 275 sterilizes the products or product packaging by breaking down micro-organisms. Ultraviolet light has a short wavelength that is harmful to microorganisms and destroys their nucleic acids. The ultraviolet radiation disrupts the microorganisms' DNA thereby killing them by eliminating their reproductive capabilities. Microorganisms cannot survive prolonged exposure to ultraviolet light.

In the embodiment illustrated in FIG. 4, the ultraviolet light module 275 is positioned so that it surrounds the inventory of products or positioned in front of, in back of, above, below, or on sides of the products. In embodiments of the present invention the ultraviolet light module 275 is disposed in a combination of positions.

Since reflection increases the effectiveness of the ultraviolet sterilization, in embodiments of the present invention the ultraviolet light module 275 further comprises metal, for example, aluminum that is used to increase ultraviolet reflection and intensity.

Although some products have been sterilized after production, the sterilization procedures are performed at periodic or intermittent times by the ultraviolet light module 275 to further ensure that the products remain sterilized.

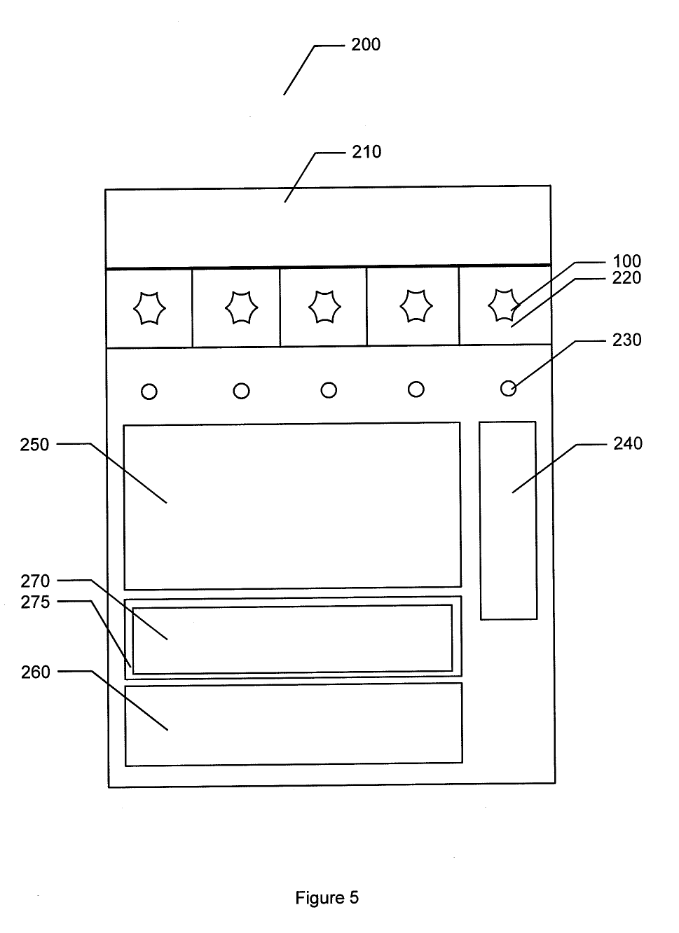

Refer to FIG. 5, which is a drawing illustrating a sanitary dispensing device with an ultraviolet light sterilization module and sterilizing area according to an embodiment of the present invention.

The embodiment illustrated in FIG. 5 is similar to the embodiment illustrated in FIG. 4, however, in the embodiment illustrated in FIG. 5 the dispensing device 200 further comprises an ultraviolet sterilization area 270.

In this embodiment the ultraviolet light module 275 is positioned in or around the ultraviolet sterilization area 270. Once a product has been purchased, the product drops into the ultraviolet sterilization area 270 from the product storage area 250 or the product display area 220 (if the product display area is acting as the product storage area). Then, the ultraviolet light module 275 performs a sterilization procedure on the product in the ultraviolet sterilization area 270. After the sterilization procedure is complete the sterilized product drops into the product retrieval area 260 and the user retrieves their purchased product.

In this embodiment all external surfaces of the product or the product packaging are sterilized. Additionally, the sterilization procedure is performed only on purchased products and at the time of purchase. Furthermore, users are additionally ensured that the item they purchased has been sterilized.

In an embodiment of the present invention the ultraviolet light module 275 is disposed in the product retrieval area 260. Once a product has been purchased, the product drops into the product retrieval area 260 and the ultraviolet light module 275 performs a sterilization procedure on the product or product packaging. A door on the product retrieval area 260 remains locked until the sterilization procedure is complete. This prevents the user from retrieving the product while the ultraviolet light module is turned on.

Additionally, a sterilization procedure in operation indicator such as a light is provided to inform users that a sterilization procedure is currently being performed and to inform users that the sterilization procedure is complete.

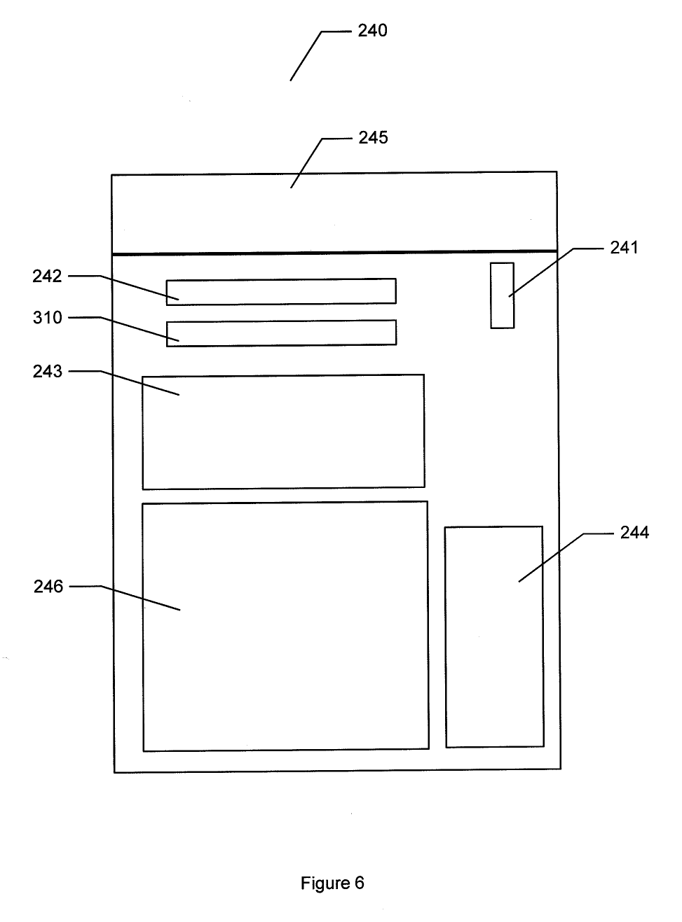

Refer to FIG. 6, which is a drawing illustrating a payment exchange area of a sanitary dispensing device according to an embodiment of the present invention.

In the embodiment illustrated in FIG. 6, the payment exchange area 240 further comprises a coupon output 310. In this embodiment of the present invention a user can purchase items that are not stored in the sanitary dispensing device. This allows more products or items to be offered for sale than need to be physically inventoried in the dispensing device.

For example, pictures, photos, videos, or advertisements are displayed on the removable display or in the product display area of the sanitary dispensing device. A user makes the selection for the desired product and a coupon is issued for the selected product. After the coupon is retrieved the user takes the coupon to a physical store or a distribution site and exchanges the coupon for the selected product or the selected product is delivered to the user's residence and the user provides the coupon upon delivery of the product.

In an embodiment of the present invention the user submits payment into the dispensing device prior to being issued a coupon for the selected item.

In another embodiment of the present invention the user submits payment to the store, distribution site, or delivery personnel along with the coupon in order to receive the selected product.

In another embodiment of the present invention a coupon is issued to a user who currently purchased a product from the dispensing device. This allows the dispensing device of the present invention to offer discounts or specials to users of the sanitary dispensing device.

In another embodiment of the present invention the sanitary dispensing device provides discounts with collective group purchasing. The product supplier, for example, purchases in bulk or operates a collective operation that allows groups of individuals to purchase items together and receive a group discount without the users having to make the purchases together as a group. The users enjoy the benefits of group purchasing without the difficulties in joining and following a group in order to make a purchase at a group discount.

For example, using the coupon issued by the sanitary dispensing device, the user is able to exchange the coupon for products at a group purchase price.

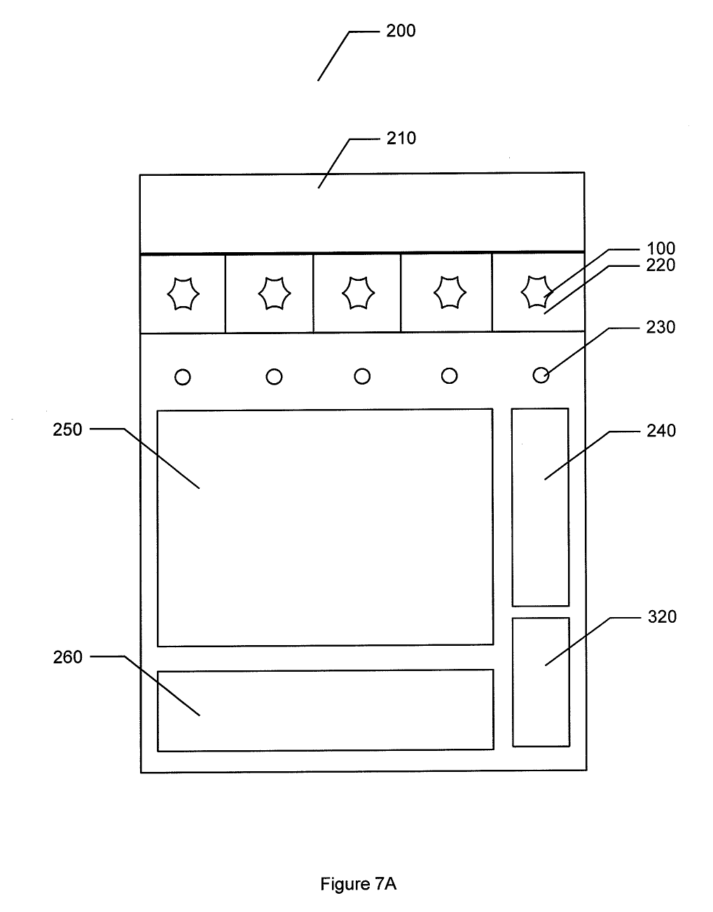

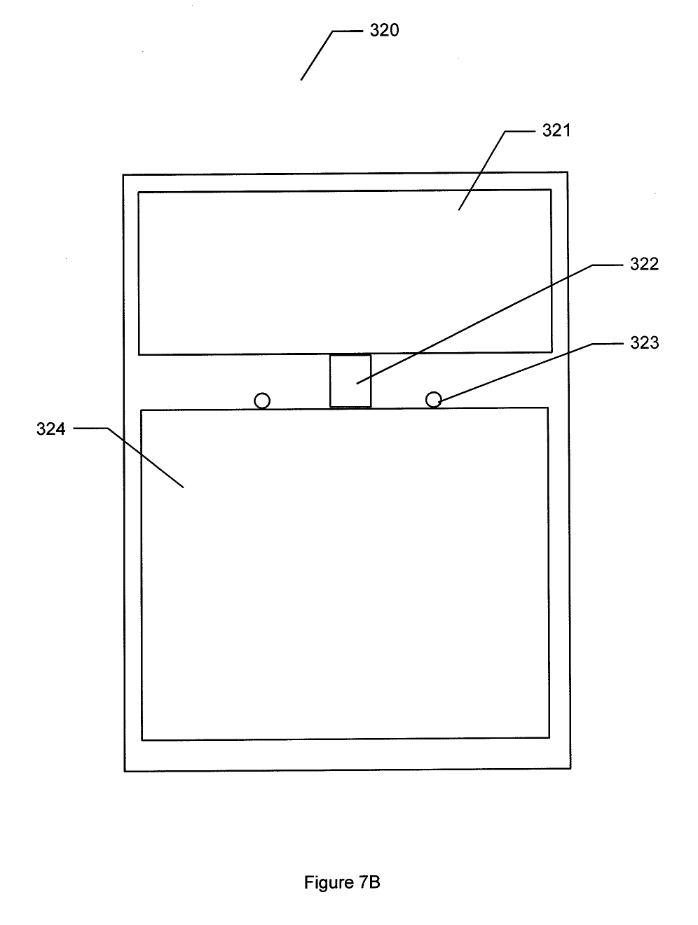

Refer to FIG. 7A, which is a drawing illustrating a sanitary dispensing device with a hands sterilization module with sterilizing area according to an embodiment of the present invention and to FIG. 7B, which is a drawing illustrating a hands sterilization module with sterilizing area according to an embodiment of the present invention.

In the embodiment illustrated in FIGS. 7A-7B, the sanitary dispensing device 200 comprises a hands sterilization module 320. The hands sterilization module 320 allows a user to sterilize their hands after purchase and prior to retrieving the purchased product.

The hands sterilization module 320 comprises a sterilizing material compartment 321, a dispenser 322, a sensor 323, and a hand sterilizing area 324.

The sterilizing material compartment 321 stores a germicidal or sterilizing gel or sterilizing liquid. The dispenser 322 is connected to the sterilizing material compartment 321 and sprays the sterilizing liquid or pumps out the sterilizing gel from the sterilizing material compartment 321.

A least one sensor 323 is provided to sense when a user inserts their hands into the hand sterilizing area 324 and signals the dispenser 322 to dispense the sterilizing gel or liquid onto the user's hands in the hand sterilizing area 324. The user simply rubs their hands and the sterilizing material sterilizes the user's hands.

In application, the user selects the product they want to purchase and inserts payment into the payment area 240 of the sanitary dispensing device 200. The payment controller signals the hands sterilizing module 320 that a purchase has been made. The user inserts their hands into the hand sterilizing area 324 and the dispenser 322 dispenses the sterilizing material to sterilize the user's hands. The user, having sterilized hands, can now retrieve their purchased sanitary product from the sanitary dispensing machine.

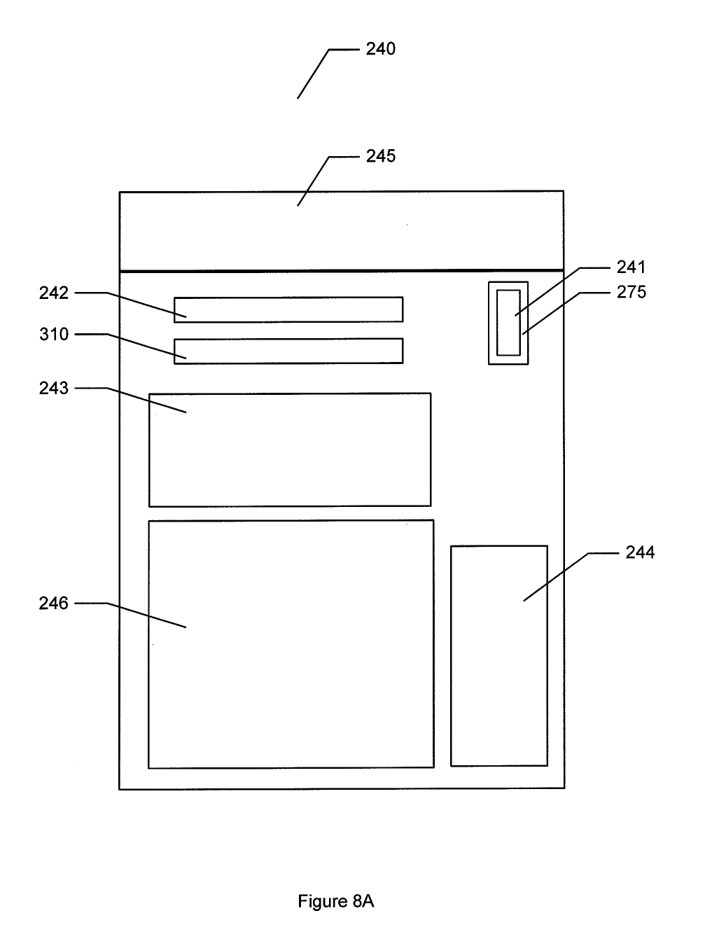

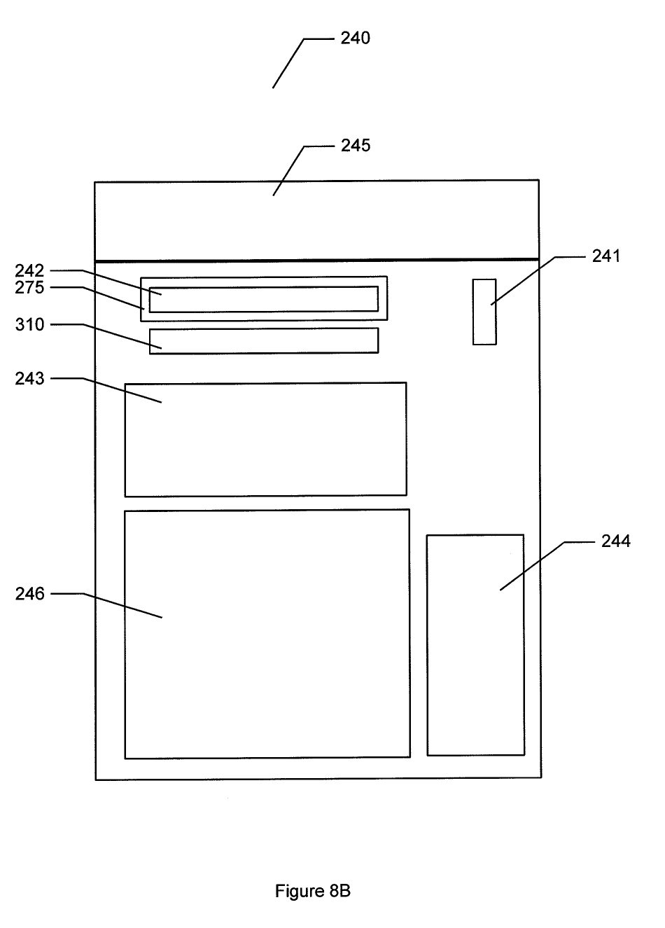

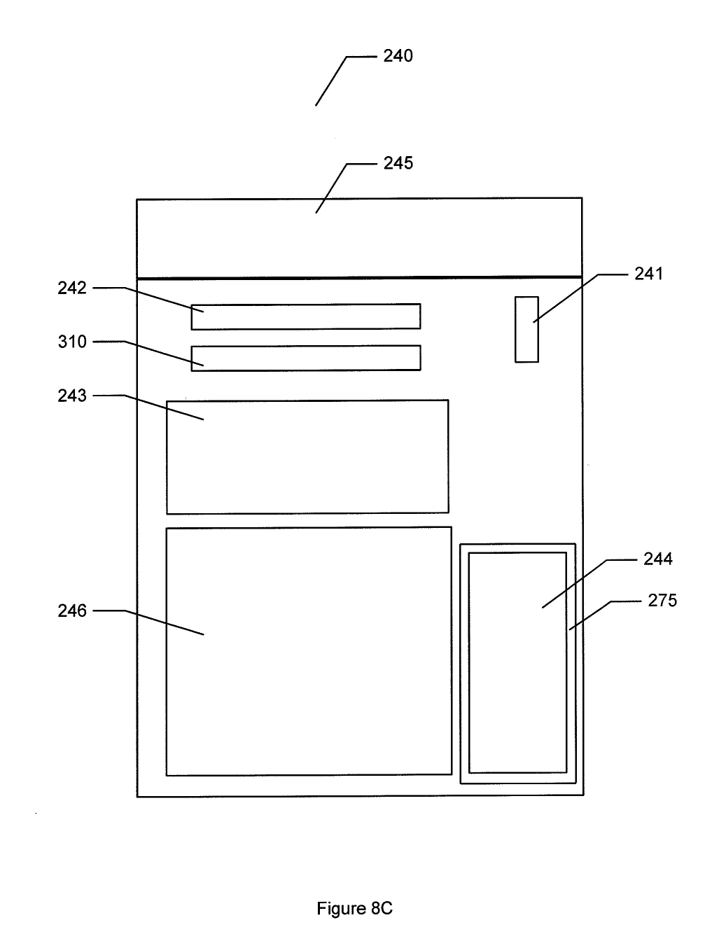

Refer to FIG. 8A, which is a drawing illustrating a sanitary dispensing device with a coin currency input ultraviolet light sterilization module according to an embodiment of the present invention.

In the embodiment illustrated in FIG. 8A, the sanitary dispensing device 200 further comprises an ultraviolet light module 275 positioned inside or around the coin currency input 241. In this embodiment the ultraviolet light module 275 is provided for performing sterilization procedures on the coins input by a user into the coin currency input 241.

The ultraviolet light module 275 comprises a lamp or a plurality of lamps, for example, a mercury-vapor lamp that emits ultraviolet light at the germicidal wavelength.

Since money is frequency passed between people, stored in unhygienic areas, touched by hands or devices, money such as coin currency or paper currency is carries and passes germs or bacteria and is usually unsanitary.

The ultraviolet light module 275 sterilizes the coins by breaking down micro-organisms. Ultraviolet light has a short wavelength that is harmful to microorganisms and destroys their nucleic acids. The ultraviolet radiation disrupts the microorganisms' DNA thereby killing them by eliminating their reproductive capabilities. Microorganisms cannot survive prolonged exposure to ultraviolet light.

In the embodiment illustrated in FIG. 8A, the ultraviolet light module 275 is positioned so that it surrounds the coin currency input 241 or positioned in front of, in back of, above, below, or on sides of the path that the coins travel upon entering the coin currency input 241. In embodiments of the present invention the ultraviolet light module 275 is disposed in a combination of positions in order to sterilize the coins after being inserted into the coin input area until the coins are stored in the dispensing device or while the coins are stored in the dispensing device.

Refer to FIG. 8B, which is a drawing illustrating a sanitary dispensing device with a paper currency input ultraviolet light sterilization module according to an embodiment of the present invention.

The embodiment illustrated in FIG. 8B is similar to the embodiment of FIG. 8A.

In the embodiment illustrated in FIG. 8B the ultraviolet light sterilization module 275 is positioned in or around the paper currency input 242 and/or positioned in the path that the paper currency travels in the dispensing device after being inserted into the paper currency input 242 and/or positioned in or around the area where the paper currency is stored in the dispensing device.

Refer to FIG. 8C, which is a drawing illustrating a sanitary dispensing device with a change return ultraviolet light sterilization module according to an embodiment of the present invention.

Similar to FIG. 8A and FIG. 8B, in the embodiment illustrated in FIG. 8C the ultraviolet light sterilization module 275 is positioned in or around the change return 244 and/or positioned in the path that the coin currency or paper currency travels in the dispensing device after being released to travel to the change return 244 as change to be returned to the user.

Utilizing the ultraviolet light sterilization module 275 of the present invention, coins and paper currency that are input into the dispensing device as payment and change returned to the user are sterilized prior to being provided to users of the sanitary dispensing device.

As a result, users of the sanitary dispensing device are ensured of a higher level of hygienic interaction than conventional dispensing device can provide.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

What is claimed is:

1. A sanitary dispensing device comprising:
a product display area for displaying at least one product;
at least one product selection switch for selecting one of the at least one product;
a payment exchange area for receiving payment in exchange for one of the at least one product;
a product retrieval area for allowing purchased products to be retrieved from the dispensing device; and
at least one ultraviolet light module for sterilizing products, product packaging, currency, or a combination of products, product packaging, and currency.

2. The sanitary dispensing device of claim 1, further comprising:
a product storage area for storing products;

3. The sanitary dispensing device of claim 1, further comprising:
a removable display for displaying product information, advertising, instructional information, or a combination of product information, advertising, and instructional information.

4. The sanitary dispensing device of claim 1, further comprising:
a hands sterilizing module for performing a sterilization procedure on a user's hands.

5. The sanitary dispensing device of claim 1, the payment exchange area comprising:
a payment display for displaying amount of money owed, amount of money received, or amount of money owed and amount of money received;
a coin currency input for receiving payment in coins;
a paper currency input for receiving payment in paper currency; and
a change return for returning change.

6. The sanitary dispensing device of claim 5, the payment exchange area further comprising:
a coupon output for providing a coupon to a user.

7. The sanitary dispensing device of claim 5, the payment exchange area further comprising:
a card reader/writer for interacting with a debit card or membership card.

8. The sanitary dispensing device of claim 3, the removable display displaying dynamic advertising based upon a user's product selection.

9. A sanitary dispensing device comprising:
a product display area for displaying products;
at least one product selection switch for selecting one of the products;
a product storage area for storing products;
a payment exchange area for receiving payment in exchange for one of the products;
a product retrieval area for allowing purchased products to be retrieved from the dispensing device; and
at least one sterilization module for sterilizing products, product packaging, currency, user's hands, or a combination of products, product packaging, currency, and user's hands.

10. The sanitary dispensing device of claim 9, further comprising:
a removable display for displaying product information, advertising, instructional information, dynamic advertising based on a user's selection of product, or a combination of product information, advertising, instructional information, and dynamic advertising based on the user's selection of product.

11. The sanitary dispensing device of claim 9, the at least one sterilization module comprising:
an ultraviolet light module for sterilizing products, product packaging, currency, or a combination of products, product packaging, and currency.

12. The sanitary dispensing device of claim 9, the payment exchange area comprising:
a payment display for displaying amount of money owed, amount of money received, or amount of money owed and amount of money received;
a coin currency input for receiving payment in coins;
a paper currency input for receiving payment in paper currency; and
a change return for returning change.

13. The sanitary dispensing device of claim 12, the payment exchange area further comprising:
a coupon output for providing a coupon to a user.

14. The sanitary dispensing device of claim 12, the payment exchange area further comprising:
a card reader/writer for interacting with a debit card or membership card.

15. The sanitary dispensing device of claim 9, the at least one sterilization module comprising:
a hands sterilizing module for performing a sterilization procedure on a user's hands.

16. A sanitary dispensing device comprising:
a product display area for displaying at least one product;
at least one product selection switch for selecting one of the at least one products;
a payment exchange area for receiving payment in exchange for one of the at least one products;
a product retrieval area for allowing purchased products to be retrieved from the sanitary dispensing device;
a removable display for displaying product information, advertising, instructional information, dynamic advertising based on a user's selection of product, or a combination of product information, advertising, instructional information, and dynamic advertising based on the user's selection of product;
at least one ultraviolet light module for sterilizing products, product packaging, currency, or a combination of products, product packaging, and currency; and
a hands sterilizing module for sterilizing a user's hands.

17. The sanitary dispensing device of claim 16, the payment exchange area comprising:
a payment display for displaying amount of money owed, amount of money received, or amount of money owed and amount of money received;
a coin currency input for receiving payment in coins;
a paper currency input for receiving payment in paper currency; and
a change return for returning change.

18. The sanitary dispensing device of claim 17, the payment exchange area further comprising:
a card reader/writer for interacting with a debit card or membership card.

19. The sanitary dispensing device of claim 17, the payment exchange area further comprising:
a coupon output for providing a coupon to a user.

20. The sanitary dispensing device of claim 16, the at least one ultraviolet light module sterilizing all coin currency and paper currency input into or output from the sanitary dispensing device.
